(19) United States
(12) Plant Patent Application Publication
(10) Pub. No.: US 2006/0294631 P1
(43) Pub. Date: Dec. 28, 2006

(54) OSTEOSPERMUM PLANT NAMED 'OSJAMLIPUR'

(50) OSTEOSPERMUM ECKLONIS; OSTEOSPERMUM

(75) Inventor: Johannes Simon Nicolaas Oud, Bovenkarspel (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/167,498

(22) Filed: Jun. 27, 2005

(51) Int. Cl. A01H 5/00

(52) U.S. Cl. PLT/360

(57) ABSTRACT

A new Osteospermum plant particularly distinguished by its very compact round plant habit, early flowering with light purple colored ray florets and blue colored disc florets.

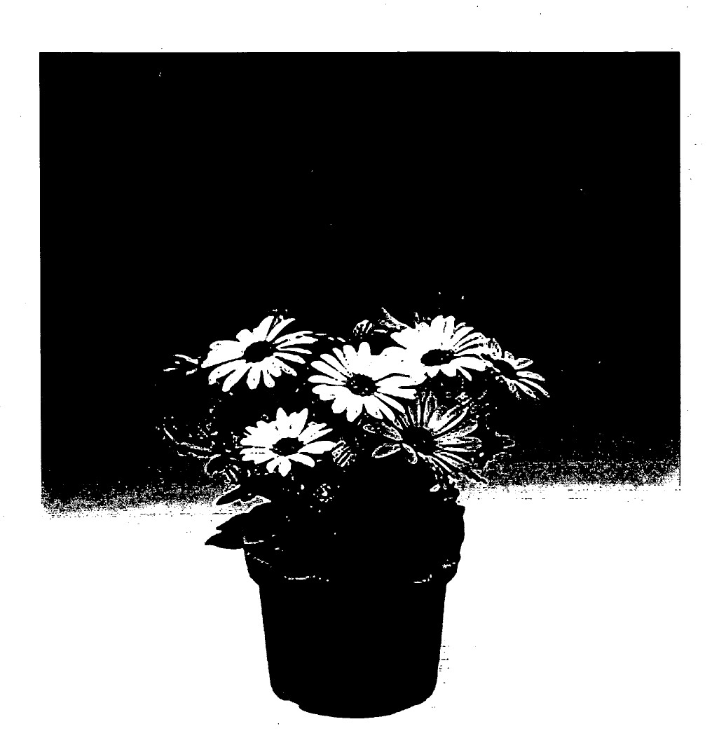

### LATIN NAME OF THE GENUS AND SPECIES OF THE PLANT CLAIMED

Osteospermum ecklonis

### VARIETAL DENOMINATION

‘Osjamlipur’

### BACKGROUND OF THE NEW PLANT

The present invention relates to a new and distinct cultivar of Osteospermum plant, botantially known as Osteospermum ecklonis. The new Osteospermum is propagated by cuttings resulting from a cross of a proprietary Osteospermum ecklonis seedling selection as the male, or pollen, parent identified as code number ‘C272’ with a proprietary Osteospermum ecklonis selection identified as code number ‘C298’ as the female, or seed, parent. ‘C272’ has a plant height of 30 cm and a plant width of 20 cm. ‘C298’ has a plant height of 18 cm and a plant width of 12 cm.

The new Osteospermum was discovered and selected as a single flowering plant within the progeny of the stated cross grown in Enkhuizen, Netherlands in 1998. Neither ‘C272’ nor ‘C298’ has been patented. The new Osteospermum plant has been repeatably asexually reproduced by cuttings in Enkhuizen, Netherlands over a period of four years. It has been found to retain its distinctive characteristics through successive propagations, and this novelty appears to be firmly fixed.

### DESCRIPTION OF THE DRAWING

This new Osteospermum plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Osteospermum plant. The data which defines these characteristics were collected from asexual reproductions carried out in Enkhuizen, Netherlands. The plant history was taken on 15 weeks old plants. Color references are primarily to the RHS Colour Chart of The Royal Horticultural Society of London. The instant plant was grown indoors in a 10.5 cm container.

TABLE 1
Comparison between the new cultivar ‘Osjamlipur’
and a similar cultivar

| | ‘Osjamlipur’ | ‘Sea side’ (PP10, 782) |
|---|---|---|
| Plant height | 18 cm | 30 cm |
| Plant width | 15 cm | 20 cm |
| Flowering response | | Opening of first flower |
| | | one week later |

The Plant

| | |
|---|---|
| Classification - Botanical | Osteospermum ecklonis |

Parentage

| | |
|---|---|
| Female parent | A seedling named ‘C298’ is one of our |
| | seedlings from our C-generation of plants |
| | bred in 1998. |
| Male parent | A seedling named ‘C272’ is one of our |
| | seedlings from our C-generation of plants |
| | bred in 1998. |

Plant Description

| | |
|---|---|
| Growth habit | Round, compact |
| Plant height | 18 cm |
| Speading area of plant | 15 cm |
| Strength | Moderate growth |
| Branching character | Freely branching, 18 lateral |
| | branches without pinching |
| Blooming period | From May until October |

The Stem

| | |
|---|---|
| Length | 9 cm |
| Diameter | 5 mm |
| Internode length | 8 mm |
| Texture | Glabrous |
| Color | RHS 137C |

The Foliage

| Shape | | Single leaves, alternate and elliptic, apex: |
|---|---|---|
| | | broadly actue base: attenuate, margin: irregular |
| | | serration |
| Number of leaves per | | 13 |
| lateral branch | | |
| Texture | | Glabrous, leathery |
| Leaf | Color-Upper side | RHS 137A |
| | Color-Under side | RHS 137B |
| | Length | Young 30 mm, mature 65 mm |
| | Width | Young 10 mm, mature 40 mm |

| | |
|---|---|
| Petiole | Absent |

The Inflorescence

| Number of inflorescences | | 90 |
|---|---|---|
| per mature plant | | |
| Inflorescence bud | | |
| Form | | Ovoid |
| Length | | 19 mm |
| Width | | 12 mm |
| Color | | RHS N79D |
| Inflorescence lastingness | | Around 5 days depending |
| on the plant | | on the temperature |
| Inflorescence | Diameter | 66 mm |
| | Disc | 10 mm |
| | Diameter | |

| Inflorescence depth | | 12 mm |
|---|---|---|
| Fragrance | | No frangrance |
| Disc | Shape | Tubular, elongated |
| florets | Apex | Five pointed |
| | Length | 8 mm |
| | Width | 1 mm |
| | Number of | 55 |
| | disk florets | |
| | per | |
| | inflorescence | |
| | Color | RHS 99B |

| Ray | Length | | 28 mm |
|---|---|---|---|
| floret | Width | | 9 mm |
| | Shape | | Capitulum with one series of ray florets |
| | Color | | |
| | Upper surface | | RHS 77C |
| | Middle surface | | RHS 77D |
| | Lower surface | | RHS N79D |
| | Number of | | 20 |
| | ray florets | | |
| | Shape | Apex | Rounded |
| | | Base | Acute |
| | | Margin | Entire |

| | |
|---|---|
| Texture | Satiny |

| | | |
|---|---|---|
| Peduncles | Length | 5 cm |
| | Texture | Sturdy, tiny hairs |
| | Color | RHS 137C |
| | Diameter | 1.5 mm |
| | Shape | Round |
| Phyllaries | Texture | Rough, tiny hairs |
| | Number of | 20 |
| | phyllaries | |
| | Color | RHS 137C |
| | Length | 10 mm |
| | Margin | Entire |
| | Width | 1 mm |
| | Apex | Acute |
| | Shape | Ligulate |
| | Color | RHS 137C both sides |

Reproductive organs

| | | |
|---|---|---|
| Androe- | Appearance | Present on disc florets only |
| cium | Shape | Oblong, five stamen fused to one |
| | Length | 2 mm |
| | Width | 1 mm |
| | Pollen color | RHS 25A |
| | Total no. | 55 |
| | of anthers | |
| Gynoe- | Appearance | Present on both ray and disc florets |
| cium | Pistil number | One pistil per ray or disc floret |
| | Pistil length | 4 mm |
| | Stigma color | RHS 77A |
| | Stigma shape | Bipartite |

Seed development
Seed development has been observed on plants
of the new Osteospermum.

| | |
|---|---|
| Amount of seeds | Moderate |
| produced | |
| Seed length | 6 mm |
| Diameter | 3 mm |
| Color | RHS 200A |
| Shape | Elliptic |

Disease resistance:

Under commercial conditions, plants of the new Osteopermum are resistant to fungal pathogens common to Osteopermum.

What is claimed is:

1. A new and distinct variety of Osteospermum plant, substantially as illustrated and described herein.
